(19) United States
(12) Patent Application Publication
(10) Pub. No.: US 2026/0083311 A1
(43) Pub. Date: Mar. 26, 2026

(54) Oral Health Monitoring Device

(71) Applicant: Colgate-Palmolive Company, New York, NY (US)

(72) Inventors: Donghui WU, Bridgewater, NJ (US); Bryan LEE, Somerset, NJ (US); Mark GUIRGUIS, Scotch Plains, NJ (US); Benny E. URBAN, Somerset, NJ (US); Hrebesh Molly SUBHASH, Somerset, NJ (US); Brian BLOCH, Hillsborough, NJ (US); Hongbing LI, Skillman, NJ (US); Patrick Christopher ANDRUS, St. Louis, MO (US); Benjamin Cooper PRIESS, Saint Charles, MO (US); Sharan AHOOJA, St. Louis, MO (US); Michael Fredrick O'BRIEN, St. Louis, MO (US); Luke Anthony SABUS, Shawnee, KS (US)

(73) Assignee: Colgate-Palmolive Company, New York, NY (US)

(21) Appl. No.: 19/110,202

(22) PCT Filed: Aug. 28, 2023
(86) PCT No.: PCT/US2023/031282

§ 371 Date: Mar. 10, 2025

Related U.S. Application Data
(60) Provisional application No. 63/408,646, filed on Sep. 21, 2022.

(51) Int. Cl. A61B 1/24; A61B 1/00; A61B 1/05

(52) U.S. Cl. CPC A61B 1/24; A61B 1/00096; A61B 1/051

(57) ABSTRACT

In one aspect, a device for imaging an oral cavity is disclosed. The device incorporates a head and an elongate body, the head having a base portion, first and second wing structures, and first and second image sensors. The first and second image sensors are mounted to the first and second wing structures and enable imaging of the user's oral cavity. The wing structures enable simultaneous imaging of multiple tooth surfaces simultaneously and may aid in positioning of the head of the device.

### CROSS-REFERENCE TO RELATED APPLICATION

This application claims the benefit of priority from U.S. Provisional Application No. 63/408,646, filed Sep. 21, 2022, the contents of which are hereby incorporated herein by reference in their entirety.

### BACKGROUND

Oral health monitoring is a key aspect of maintaining long term oral health. Much of oral health monitoring is currently done via inspections by an oral health professional such as a dentist or hygienist. However, these inspections are often limited to visual inspection using only mirrors and fail to allow clear imaging of all oral surfaces. Improvements have been made in the realm of optical and x-ray imaging, allowing imaging of a greater portion of the user's teeth. However, many current imaging devices suffer from a need for a high degree of skill to effectively image a patient's oral cavity. Furthermore, current devices are often time consuming to operate and cannot be used by the patient. Therefore, there is a need to develop improved imaging systems which more efficiently image a patient's oral cavity while reducing the required skill level.

### BRIEF SUMMARY

The invention is directed to devices for imaging a patient's oral cavity to monitor oral health.

In one aspect, a device for oral health monitoring includes an elongate body extending along a longitudinal axis and a head coupled to the elongate body. The head has a base portion, the base portion coupled to the elongate body. The head further has first and second wing structures extending from the base portion. In addition, the head has a first image sensor coupled to the first wing structure, the first image sensor having a first sensing axis normal to an imaging surface of the first imaging sensor. The head also has a second image sensor coupled to the second wing structure, the second image sensor having a second sensing axis normal to an imaging surface of the second imaging sensor. The first and second sensing axes of the first and second image sensors intersect.

In another aspect, a device for oral health monitoring includes an elongate body extending along a longitudinal axis and a head coupled to the elongate body. The head has a base portion, the base portion coupled to the elongate body. The head further has first and second wing structures extending from the base portion. The first wing structure has an inner surface and the second wing structure has an inner surface. In addition, the head has a first image sensor coupled to the first wing structure and a second image sensor coupled to the second wing structure. The inner surfaces of the first and second wing structures are non-parallel.

In yet another aspect, a device for oral health monitoring includes an elongate body extending along a longitudinal axis and a head coupled to the elongate body. The head has a base portion, the base portion coupled to the elongate body. The head further has first and second wing structures extending from the base portion. Each of the first and second wing structures has an inner surface. In addition, the head has a first image sensor coupled to the first wing structure and a second image sensor coupled to the second wing structure. A tooth guide extends from the base portion, the tooth guide configured to engage a tooth of a user and isolate the base portion from the tooth of the user.

### BRIEF DESCRIPTION OF THE DRAWINGS

The present invention will become more fully understood from the detailed description and the accompanying drawings, wherein:

FIG. 1 is a perspective view of an oral health monitoring device.

FIG. 2 is a perspective view of a portion of the oral health monitoring device of FIG. 1.

FIG. 3 is a front view of the portion of the oral health monitoring device.

FIG. 4 is a rear view of the portion of the oral health monitoring device.

FIG. 5 is a left side view of the portion of the oral health monitoring device.

FIG. 6 is a right side view of the portion of the oral health monitoring device.

FIG. 7 is a top view of the portion of the oral health monitoring device.

FIG. 8 is a bottom view of the portion of the oral health monitoring device.

FIG. 9 is a cross-sectional view of the portion of the oral health monitoring device, taken along line IX-IX of FIG. 3.

FIG. 10 is a perspective view of a second embodiment of a portion of the oral health monitoring device as may be used in the oral health monitoring device of FIG. 1.

FIG. 11 is a front view of the portion of the oral health monitoring device.

FIG. 12 is a rear view of the portion of the oral health monitoring device.

FIG. 13 is a left side view of the portion of the oral health monitoring device.

FIG. 14 is a right side view of the portion of the oral health monitoring device.

FIG. 15 is a top view of the portion of the oral health monitoring device.

FIG. 16 is a bottom view of the portion of the oral health monitoring device.

FIG. 17 is a cross-sectional view of the portion of the oral health monitoring device, taken along line XVII-XVII of FIG. 11.

FIG. 18 is a front view of a third embodiment of a portion of the oral health monitoring device as may be used in the oral health monitoring device of FIG. 1.

FIG. 19 is a right side view of the portion of the oral health monitoring device.

FIG. 20 is a top view of the portion of the oral health monitoring device.

FIG. 21 is a perspective view of a fourth embodiment of a portion of the oral health monitoring device as may be used in the oral health monitoring device of FIG. 1.

FIG. 22 is a top view of a fifth embodiment of a portion of the oral health monitoring device as may be used in the oral health monitoring device of FIG. 1.

FIG. 23A is a perspective view of a sixth embodiment of an oral health monitoring device.

FIG. 23B is an upper right perspective view of a head portion of the oral health monitoring device with the sleeve omitted.

FIG. 23C is an upper left perspective view of the head portion of the oral health monitoring device with the sleeve omitted.

FIG. 24 is a cross-section view of the oral health monitoring device taken along line XXIVA-XXIVA of FIG. 23.

FIG. 25 is a front view of the oral health monitoring device of FIG. 23.

FIG. 26 is a left side view of the oral health monitoring device of FIG. 23.

FIG. 27 is a rear view of the oral health monitoring device of FIG. 23.

FIG. 28 is a top view of the oral health monitoring device of FIG. 23.

FIG. 29A is a perspective view of a portion of the oral health monitoring device without a sleeve.

FIG. 29B is a perspective view of a portion of the oral health monitoring device with a first sleeve.

FIG. 29C is a perspective view of a portion of the oral health monitoring device with a first sleeve.

FIG. 30 is a perspective view of a sleeve of the sixth embodiment of the oral health monitoring device of FIG. 23.

FIG. 31 is a lower rear perspective view of the sleeve of FIG. 30.

FIG. 32 is a front view of the sleeve of FIG. 30.

FIG. 33 is a rear view of the sleeve of FIG. 30.

FIG. 34 is a right view of the sleeve of FIG. 30.

FIG. 35 is a left view of the sleeve of FIG. 30.

FIG. 36 is a top view of the sleeve of FIG. 30.

FIG. 37 is a bottom view of the sleeve of FIG. 30.

FIG. 38 is a cross-section view of the sleeve of FIG. 30 taken along line XXX-XXX of FIG. 32.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

The invention described herein relates to an oral health monitoring device configured to image the oral cavity of a patient. In some implementations, the oral health monitoring device is configured to image teeth and gums of a patient. In yet other implementations, the oral health monitoring device utilizes visible light and images a tooth and a portion of the surrounding gum tissue substantially simultaneously. Optionally, this visible light may be white light. In other implementations, the oral health monitoring device utilizes UV light (approximately 350-400 nm wavelength) and images a tooth and a portion of the surrounding gum tissue substantially simultaneously in fluorescence mode. The white light and UV light sources may be used to illuminate the oral surfaces in sequence for alternating image frames. In another embodiment, users may complete a full mouth scan of white light, then switch to UV light source for a second scan for fluorescence mode. Preferably, the device utilizes one or more image sensors and features which assist with positioning of the image sensors with respect to the teeth and gums.

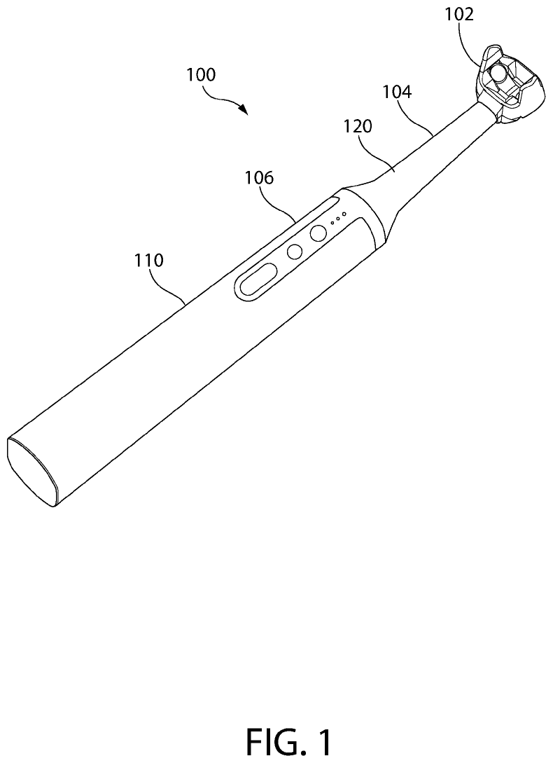

Referring to FIG. 1, an oral health monitoring device 100 is illustrated in accordance with an embodiment of the present invention. In the exemplified embodiment, the device 100 generally has a body 110 and a scan head 120 that is detachably coupled to the body 110. More specifically, the body 110 has a handle portion 106 that is configured for gripping and handling by a user. The scan head 120 has an elongate body 104 and a head 102. The scan head 120 may be detachably coupled to the body 110 with a friction/interference fit or via mechanical interaction, such as the scan head 120 having a protuberance or recess that matches with a recess or protuberance on the body 110. Various techniques for coupling a scan head 120 to a body 110 of a toothbrush 100 are known and could be used in accordance with the invention described herein (i.e., magnetic, mechanical, interference, screw threads, protuberance/detent, or the like). The scan head 120 and the body 110 are illustrated generically and the invention is not to be limited by the shape, size, and/or geometry of these components. Optionally, the scan head 120 and the body 110 may be made unitary such that the scan head 120 is not detachably coupled to the body 110.

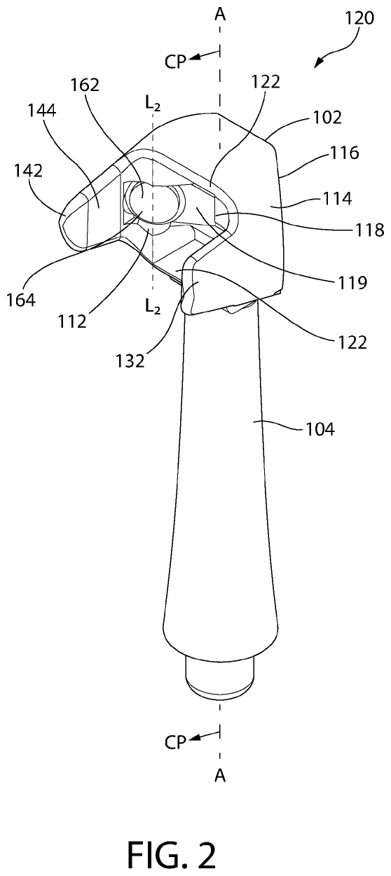

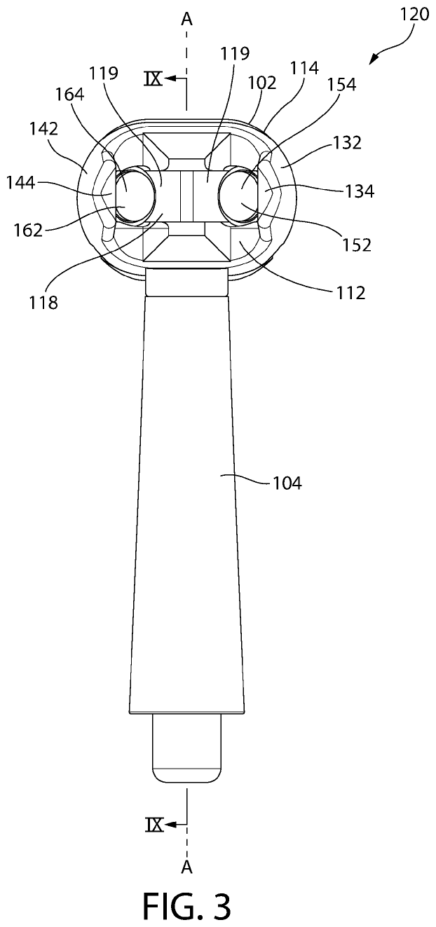

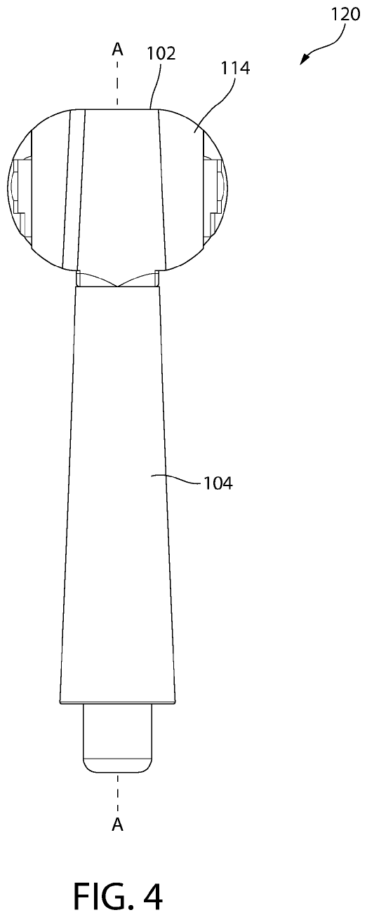

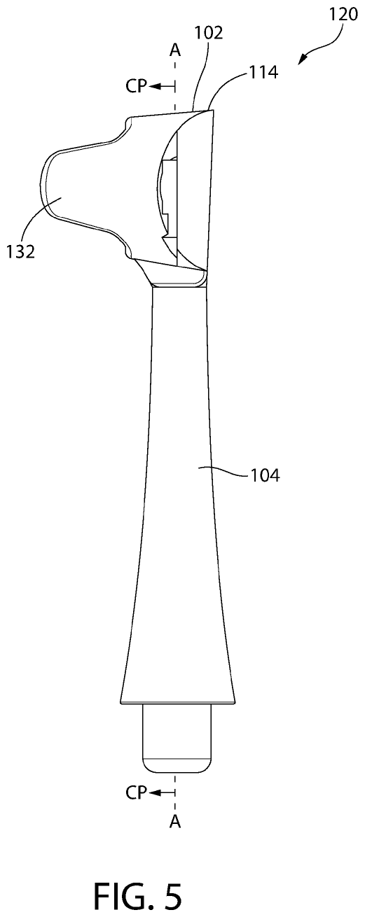

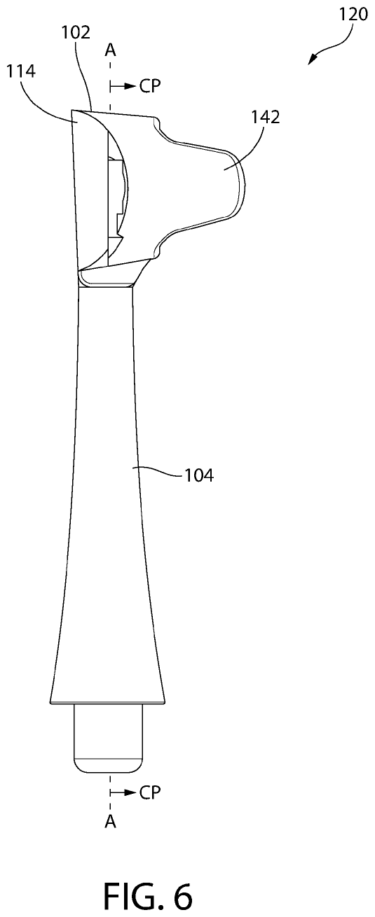

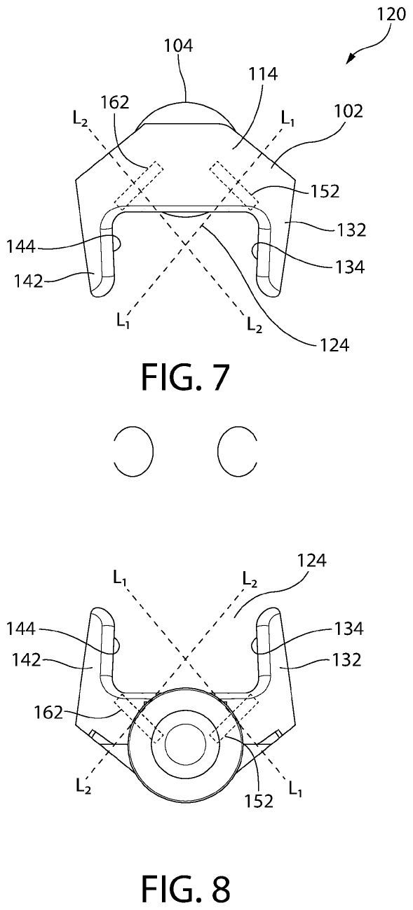

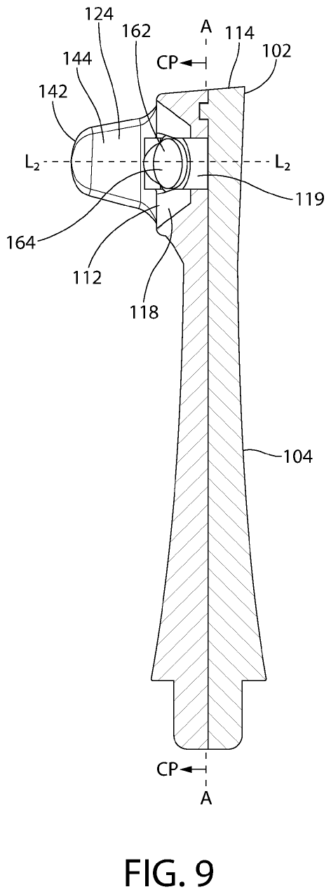

FIGS. 2-9 illustrate the scan head 120 of the device 100. As discussed above, the scan head 120 has an elongate body 104 and a head 102. The elongate body 104 extends along a longitudinal axis A-A. A central plane CP extends through the head 102, the central plane CP defined by the longitudinal axis A-A and extending perpendicular to a front surface 112 of a base portion 114 of the head 102.

The base portion 114 also has a rear surface 116 opposite the front surface 112. A first wing structure 132 and a second wing structure 142 extend from the base portion 114. In the present embodiment, the first and second wing structures 132, 142 may be symmetrical about the central plane CP. Otherwise stated, the first and second wing structures 132, 142 are mirrored about the central plane CP. In other embodiments, the first and second wing structures 132, 142 may not be symmetrical about the central plane CP.

The base portion 114 is directly coupled to the elongate body 104. The base portion 114 serves as a central structural feature from which the first and second wing structures 132, 142 extend. A first image sensor 152 is coupled to an inner surface 134 of the first wing structure 132. A second image sensor 162 is coupled to an inner surface 144 of the second wing structure 142. The first and second image sensors 152, 162 are image sensors configured to image optical light. In other embodiments, the first and second image sensors may be sensitive to X-rays, infrared light, UV light, or any other frequency band useful for monitoring oral health.

The first and second image sensors 152, 162 each have imaging surfaces 154, 164. The imaging surfaces 154, 164 may be planar or may convex or concave. Optionally, the imaging surfaces 154, 164 may be lenses or protective windows covering the first and second image sensors 152, 162. A first sensing axis L₁-L₁ is normal to the imaging surface 154 of the first image sensor 152. A second sensing axis L₂-L₂ is normal to the imaging surface 164 of the second image sensor 162. Where the imaging surfaces 154, 164 are concave or convex, the first and second sensing axes L₁-L₁, L₂-L₂ are normal to a plane which is tangent to the imaging surfaces 154, 164 at a minimum or maximum point (as applicable depending on the curvature being concave or convex). The first and second sensing axes L₁-L₁, L₂-L₂ intersect. The first and second sensing axes L₁-L₁, L₂-L₂ intersect at the central plane CP.

The base portion 114 may also have a cavity 118, the cavity 118 being recessed with respect to the front surface 112 of the base portion 114. Optionally, the first and second image sensors 152, 162 may be partially located within the cavity 118. The image sensors 152, 162 may be mounted such that they are coupled to both the inner surfaces 134, 144 of the first and second wing structures 132, 142 and an inner surface 119 of the cavity 118. In alternate embodiments, the first and second image sensors 152, 162 may be coupled exclusively to the inner surfaces 134, 144 of the first and second wing structures 132, 142 or may be coupled exclusively to the inner surface 119 of the cavity 118. Optionally, the image sensors 152, 162 may be recessed on the inner surfaces 134, 144 or the image sensors 152, 162 may be flush mounted or protruding from the inner surfaces 134, 144 of the first and second image sensors 152, 162.

The base portion 114 incorporates tooth guides 122. The tooth guides 122 are located on opposite sides of the cavity 118 and extend from the front surface 112 of the base portion 114. Optionally, the tooth guides 122 may form a part of the front surface 112. The tooth guides 122 are configured to engage a user's teeth and maintain a spacing between the user's teeth and the first and second image sensors 152, 162. The desired spacing between the user's teeth and the first and second image sensors 152, 162 may be selected to ensure that the first and second image sensors 152, 162 are positioned at an appropriate focal distance. Optionally, the first and second wing structures 132, 142 may also be used to maintain the desired spacing instead of the tooth guides 122. It is also conceived that the first and second image sensors 152, 162 may also contact the user's oral tissues and maintain the desired spacing. Thus, the shape of the image sensors 152, 162 may be altered to ensure that the desired spacing is achieved. Optionally, the tooth guides 122 may be omitted. Optionally, one or more than two tooth guides 122 may be utilized. In yet other optional configurations, the tooth guides 122 may incorporate a notch to receive a tip of the incisors. This may aid in centering the scan head 120 during use.

The tooth guides 122 may be symmetric about the central plane CP or may be non-symmetric about the central plane CP. Optionally, the tooth guides 122 extend along the central plane CP. In the present embodiment, the inner surfaces 134, 144 of the first and second wing structures 132, 142 may be non-parallel. In other implementations, the inner surfaces 134, 144 may be parallel. The inner surfaces 134, 144 are symmetrical about the central plane CP, but in other implementations the inner surfaces 134, 144 may not be symmetrical about the central plane CP. When viewed from the top or bottom of the scan head 120, the base portion 114 and the first and second wing structures 132, 142 form a tooth passageway 124 configured to receive one or more teeth of a user. The tooth passageway 124 has a U shape in cross-section.

Optionally, the tooth guides 122 and the wing structures 132, 142 may be formed at least in part of soft materials such as elastomers to provide a comfortable interface between the scan head 120 and the user's teeth and gums. Optionally, the soft material may be a thermoplastic elastomer (“TPE”) which may form an entirety of the tooth guides 122 or wing structures 132, 142. In yet other configurations, soft material may be used to form only a portion of the tooth guides 122 or the wing structures 132, 142.

The first and second image sensors 152, 162 may incorporate lights to illuminate the user's oral tissues. Optionally, the lights may be ring lights forming a perimeter of the first and second image sensors 152, 162. In other configurations, the lights may be embedded in the wing structures 132, 142 and physically separate from the remainder of the first and second image sensors 152, 162. Optionally, the lights may be LEDs (light emitting diodes) which may be directly mounted on the wing structures 132, 142 or may be embedded, with light pipes or light guides used to direct light from the LEDs to the user's oral tissues. The lights or the light guides may also incorporate a diffuser which may be embedded within the wing structures 132, 142.

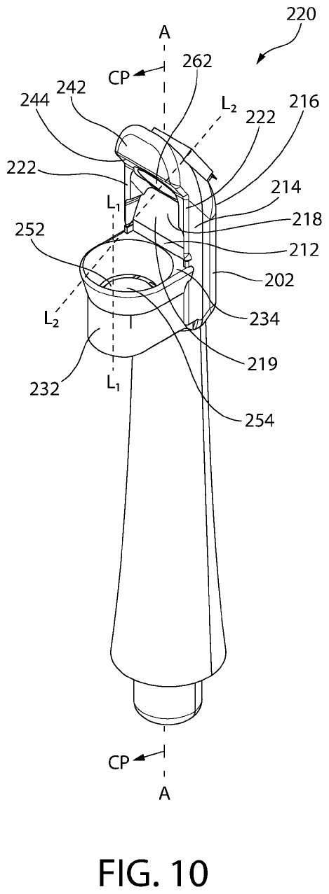

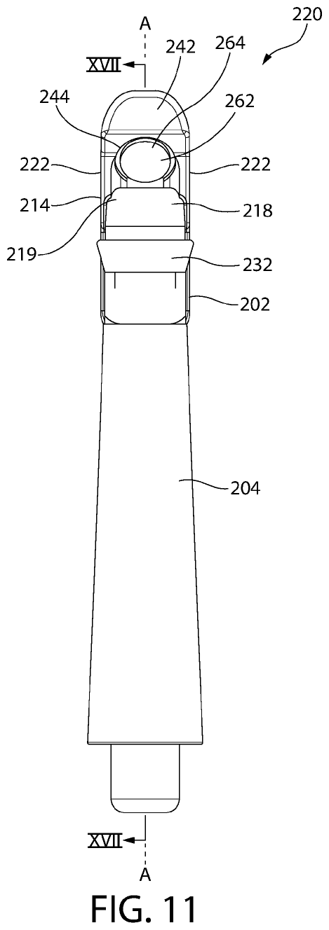

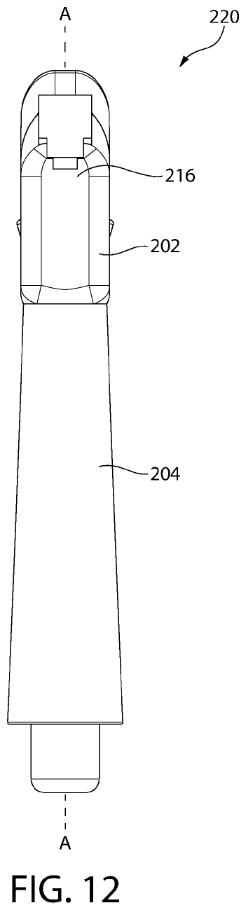

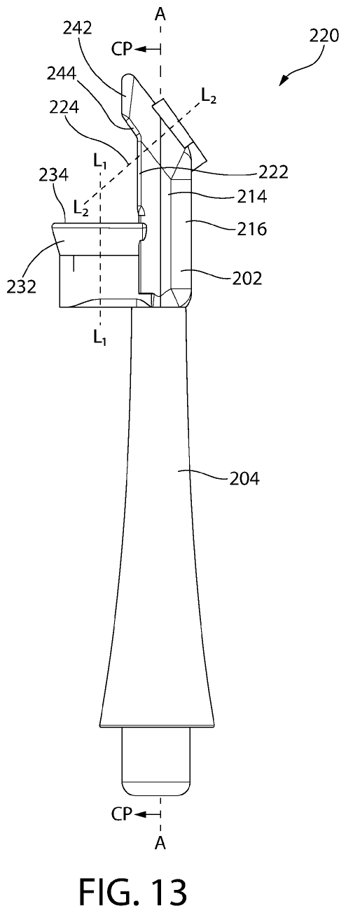

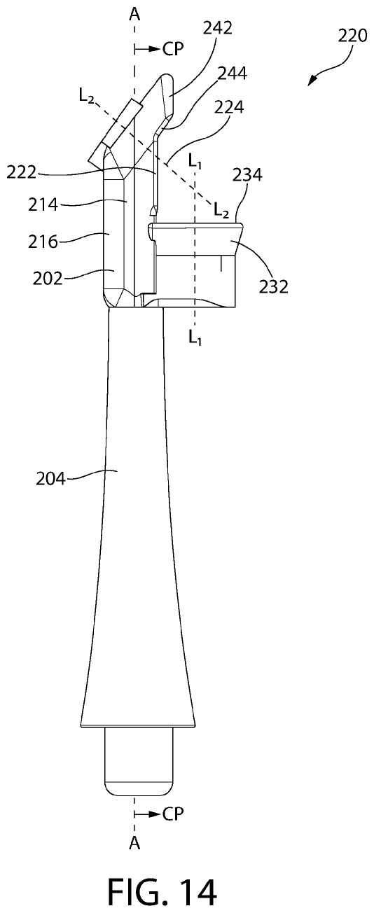

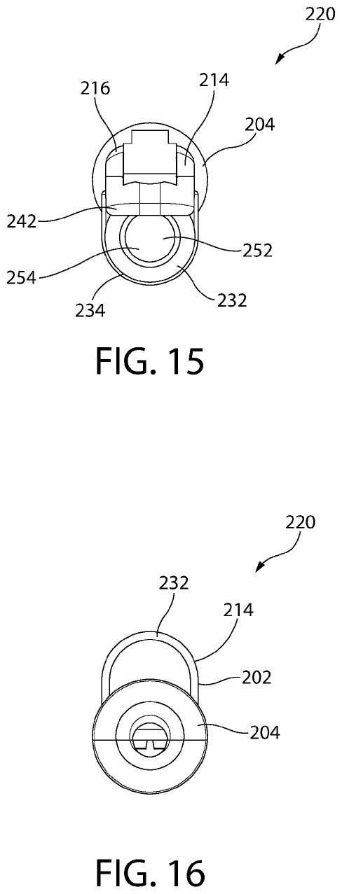

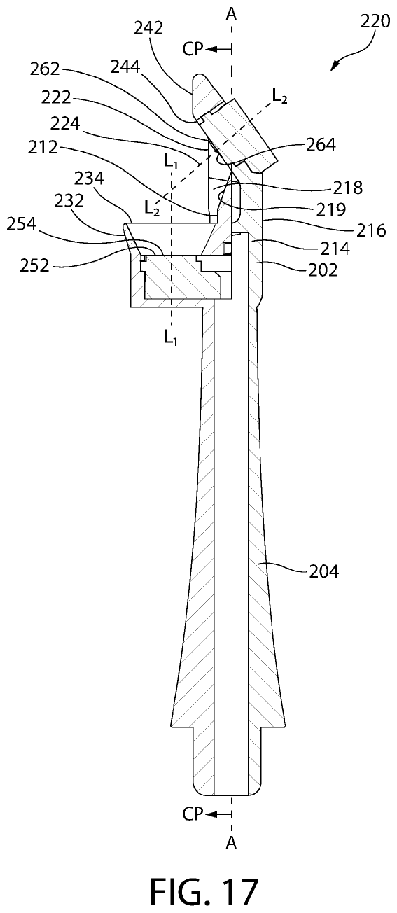

Turning to FIGS. 10-17, a second embodiment of a scan head 220 is illustrated. The scan head 220 may be utilized with the body 110 shown in FIG. 1 or may be formed with an integral handle as desired. The scan head 220 has an elongate body 204 and a head 202. The elongate body 204 extends along a longitudinal axis A-A. A central plane CP extends through the head 202, the central plane CP defined by the longitudinal axis A-A and extending perpendicular to a front surface 212 of a base portion 214 of the head 202.

The base portion 214 also has a rear surface 216 opposite the front surface 212. A first wing structure 232 and a second wing structure 242 extend from the base portion 214. In the present embodiment, the first wing structure 232 may be symmetrical about the central plane CP and the second wing structure 242 may also be symmetrical about the central plane CP. Otherwise stated, each of the first and second wing structures 232, 242 extend from the front surface 212 of the base portion 214 and extend laterally with respect to the central plane CP and the longitudinal axis A-A. The first wing structure 232 extends substantially perpendicular to the longitudinal axis A-A while the second wing structure 242 extends at an oblique angle to the longitudinal axis.

The base portion 214 is directly coupled to the elongate body 204. The base portion 214 serves as a central structural feature from which the first and second wing structures 232, 242 extend. A first image sensor 252 is coupled to an inner surface 234 of the first wing structure 232. A second image sensor 262 is coupled to an inner surface 244 of the second wing structure 242. The first and second image sensors 252, 262 are image sensors configured to image optical light. In other embodiments, the first and second image sensors may be sensitive to X-rays, infrared light, UV light, or any other frequency band useful for monitoring oral health. Optionally, the first and second image sensors 252, 262 may be recessed within the inner surfaces 234, 244 of the first and second wing structures 232, 242.

The first and second image sensors 252, 262 each have imaging surfaces 254, 264. The imaging surfaces 254, 264 may be planar or may convex or concave. A first sensing axis L₁-L₁ is normal to the imaging surface 254 of the first image sensor 252. A second sensing axis L₂-L₂ is normal to the imaging surface 264 of the second image sensor 262. Where the imaging surfaces 254, 264 are concave or convex, the first and second sensing axes L₁-L₁, L₂-L₂ are normal to a plane which is tangent to the imaging surfaces 254, 264 at a minimum or maximum point (as applicable depending on the curvature being concave or convex). The first and second sensing axes L₁-L₁, L₂-L₂ intersect. The first and second sensing axes L₁-L₁, L₂-L₂ extend along the central plane CP. Optionally, the first sensing axis L₁-L₁ may be parallel to the longitudinal axis A-A.

The first and second sensing axes L₁-L₁, L₂-L₂ may intersect at 90 degrees. In other implementations, the first and second sensing axes L₁-L₁, L₂-L₂ may intersect at less than or greater than 90 degrees. Preferably, the first and second sensing axes may intersect at between 60 and 120 degrees. Similarly, the imaging surfaces 254, 264 may also be arranged such that they extend from the base portion 214 and are angled between 0 and 10 degrees with respect to one another. Optionally, the imaging surfaces 254, 264 may extend from the base portion 214 such that they are closer together with increasing distance from the base portion 214. In other implementations, the imaging surfaces 254, 264 may be further apart with increasing distance from the base portion 214. In those implementations, the imaging surfaces 254, 264 may be angled at an angle of 20 to 90 degrees.

The base portion 214 may also have a cavity 218, the cavity 218 being recessed with respect to the front surface 212 of the base portion 214. Optionally, the first and second image sensors 252, 262 may be partially located within the cavity 218. One or both of the image sensors 252, 262 may be mounted such that they are coupled to both the inner surfaces 234, 244 of the first and second wing structures 232, 242 and an inner surface 219 of the cavity 218. In alternate embodiments, the first and second image sensors 252, 262 may be coupled exclusively to the inner surfaces 234, 244 of the first and second wing structures 232, 242 or may be coupled exclusively to the inner surface 219 of the cavity 218. Optionally, the image sensors 252, 262 may be recessed on the inner surfaces 234, 244 or the image sensors 252, 262 may be flush mounted or protruding from the inner surfaces 234, 244 of the first and second image sensors 252, 262.

The base portion 214 incorporates tooth guides 222. The tooth guides 222 are located on opposite sides of the cavity 218 and extend from the front surface 212 of the base portion 214. Optionally, the tooth guides 222 may form a part of the front surface 212. The tooth guides 222 are configured to engage a user's teeth and maintain a spacing between the user's teeth and the first and second image sensors 252, 262. The desired spacing between the user's teeth and the first and second image sensors 252, 262 may be selected to ensure that the first and second image sensors 252, 262 are positioned at an appropriate focal distance. Optionally, the first and second wing structures 232, 242 may also be used to maintain the desired spacing instead of the tooth guides 222. Optionally, the tooth guides 222 may be omitted. Optionally, one or more than two tooth guides 222 may be utilized.

The tooth guides 222 may extend along the central plane CP and extend transverse to the central plane CP. In the present embodiment, the inner surfaces 234, 244 of the first and second wing structures 232, 242 may be non-parallel. Optionally, the inner surface 234 may be perpendicular to the longitudinal axis A-A and perpendicular to the central plane CP. In other implementations, the inner surfaces 234, 244 may be parallel. When viewed from the top or bottom of the scan head 220, the base portion 214 and the first and second wing structures 232, 242 form a tooth passageway 224 configured to receive one or more teeth of a user.

During use, the scan head 220 may be configured to take a series of images as the user moves the scan head 220 along the teeth. These images may be stitched together using image processing techniques to provide a view of all surfaces of the user's teeth and optionally a portion of the adjacent gums. Stitching may be performed via any known technique. It is further conceived that stitching may occur using images which are obtained at different times, thus enabling stitching of images capturing a plurality of the user's teeth. A plurality of images may also be used to capture the teeth from a variety of perspectives, allowing the creation of a 3D model of the user's teeth.

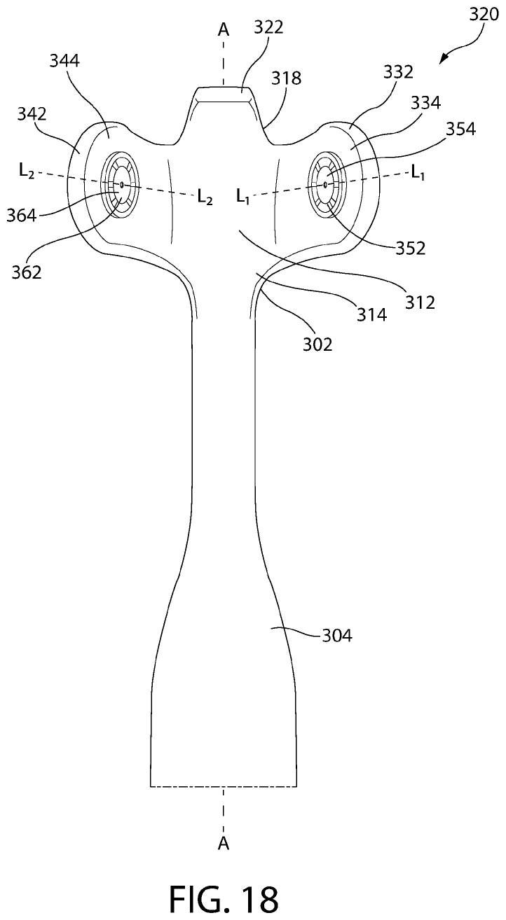

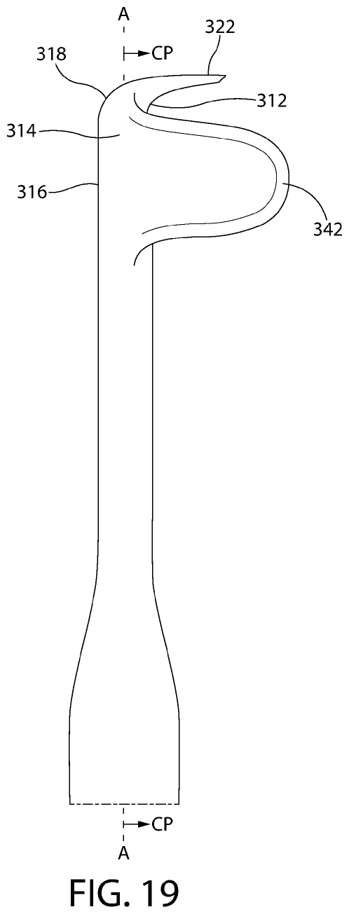

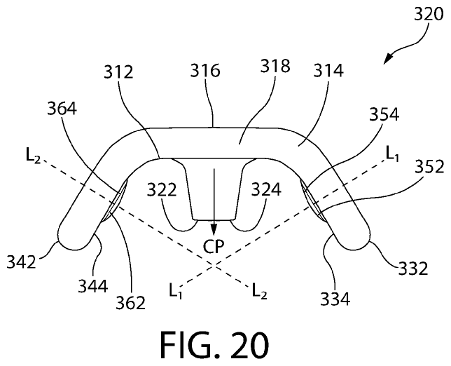

Turning to FIGS. 18-20, a third embodiment of a scan head 320 is illustrated. The scan head 320 may be utilized with the body 110 shown in FIG. 1 or may be formed with an integral handle as desired. The scan head 320 has an elongate body 304 and a head 302. The elongate body 304 extends along a longitudinal axis A-A. A central plane CP extends through the head 302, the central plane CP defined by the longitudinal axis A-A and extending perpendicular to a front surface 312 of a base portion 314 of the head 302.

The base portion 314 also has a rear surface 316 opposite the front surface 314. A first wing structure 332 and a second wing structure 342 extend from the base portion 314. In the present embodiment, the first and second wing structures 332, 342 may be symmetrical about the central plane CP. Otherwise stated, the first and second wing structures 332, 342 are mirrored about the central plane CP.

The base portion 314 is directly coupled to the elongate body 304. The base portion 314 serves as a central structural feature from which the first and second wing structures 332, 342 extend. A first image sensor 352 is coupled to an inner surface 334 of the first wing structure 332. A second image sensor 362 is coupled to an inner surface 344 of the second wing structure 342. The first and second image sensors 352, 362 are image sensors configured to image optical light. In other embodiments, the first and second image sensors may be sensitive to X-rays, visible light, infrared light, UV light, or any other frequency band useful for monitoring oral health.

The first and second image sensors 352, 362 each have imaging surfaces 354, 364. The imaging surfaces 354, 364 may be planar or may convex or concave. A first sensing axis L₁-L₁ is normal to the imaging surface 354 of the first image sensor 352. A second sensing axis L₂-L₂ is normal to the imaging surface 364 of the second image sensor 362. Where the imaging surfaces 354, 364 are concave or convex, the first and second sensing axes L₁-L₁, L₂-L₂ are normal to a plane which is tangent to the imaging surfaces 354, 364 at a minimum or maximum point (as applicable depending on the curvature being concave or convex). The first and second sensing axes L₁-L₁, L₂-L₂ intersect. The first and second sensing axes L₁-L₁, L₂-L₂ intersect at the central plane CP.

The base portion 314 incorporates a tooth guide 322. The tooth guide 322 is located at a distal end 318 of the base portion 314. The tooth guide 322 extends from the front surface 312 of the base portion 314. The tooth guide 322 may extend along the central plane CP and be symmetric about the central plane CP. In the present embodiment, the inner surfaces 334, 344 of the first and second wing structures 332, 342 may be non-parallel. The inner surfaces 334, 344 are symmetrical about the central plane CP. When viewed from the top or bottom of the scan head 320, the base portion 314 and the first and second wing structures 332, 342 form a tooth passageway 324 configured to receive one or more teeth of a user. The tooth passageway 324 has a generally U or V shape in cross-section.

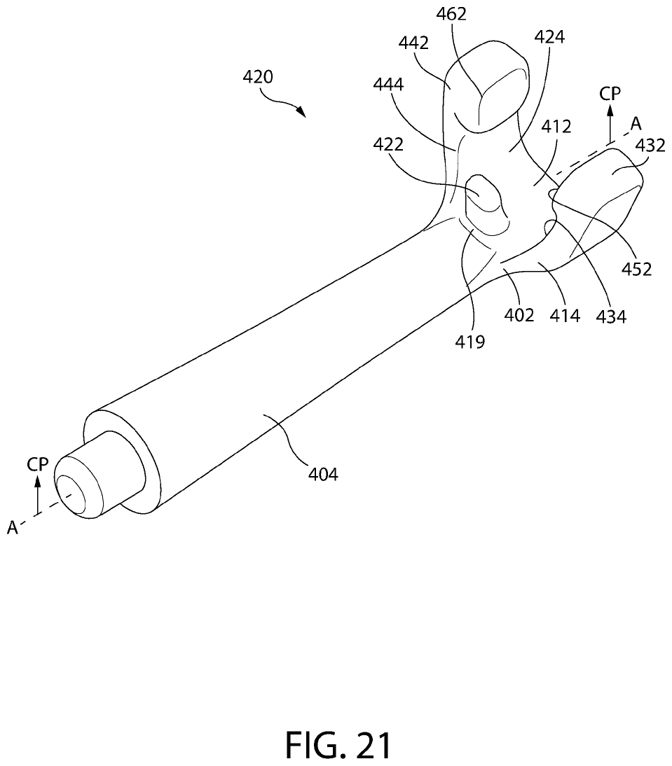

Turning to FIG. 21, a fourth embodiment of a scan head 420 is illustrated. The scan head 420 may be utilized with the body 110 shown in FIG. 1 or may be formed with an integral handle as desired. The scan head 420 has an elongate body 404 and a head 402. The elongate body 404 extends along a longitudinal axis A-A. A central plane CP extends through the head 402, the central plane CP defined by the longitudinal axis A-A and extending perpendicular to a front surface 412 of a base portion 414 of the head 402.

The base portion 414 also has a rear surface 416 opposite the front surface 414. A first wing structure 432 and a second wing structure 442 extend from the base portion 414. In the present embodiment, the first and second wing structures 432, 442 may be symmetrical about the central plane CP. Otherwise stated, the first and second wing structures 432, 442 are mirrored about the central plane CP.

The base portion 414 is directly coupled to the elongate body 404. The base portion 414 serves as a central structural feature from which the first and second wing structures 432, 442 extend. A first image sensor 452 is coupled to an inner surface 434 of the first wing structure 432. A second image sensor 462 is coupled to an inner surface 444 of the second wing structure 442. The first and second image sensors 452, 462 are image sensors configured to image optical light. In other embodiments, the first and second image sensors may be sensitive to X-rays, visible light, infrared light, UV light, or any other frequency band useful for monitoring oral health.

The base portion 414 incorporates a tooth guide 422. The tooth guide 422 is located at a proximal end 419 of the base portion 414. The tooth guide 422 extends from the front surface 412 of the base portion 414. The tooth guide 422 may extend along the central plane CP and be symmetric about the central plane CP. In the present embodiment, the inner surfaces 434, 444 of the first and second wing structures 432, 442 may be non-parallel. The inner surfaces 434, 344 are symmetrical about the central plane CP. The base portion 414 and the first and second wing structures 432, 442 form a tooth passageway 424 configured to receive one or more teeth of a user. The tooth passageway 424 has a generally U or V shape in cross-section.

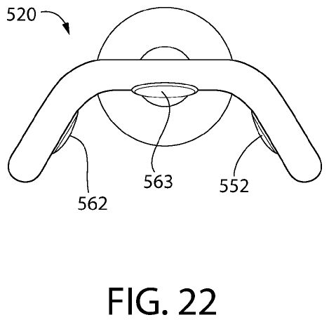

FIG. 22 illustrates a fifth embodiment of a scan head 520. The scan head 520 incorporates three image sensors 552, 562, 563. Optionally, the third image sensor 563 may be utilized to image grinding surfaces of the premolars and molars rather than rely on portions of images taken by the first and second image sensors 552, 562. This may deliver a superior quality image of the grinding surface.

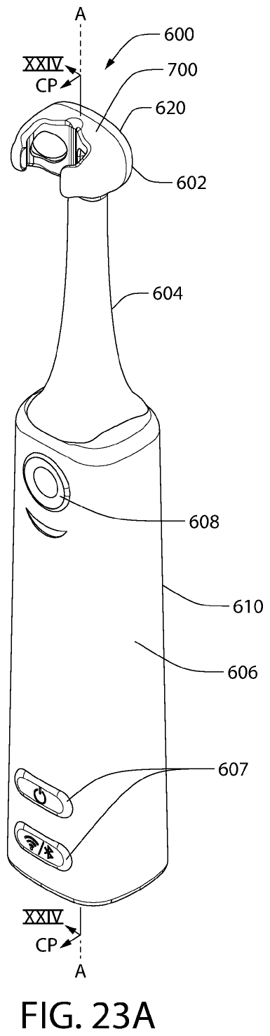

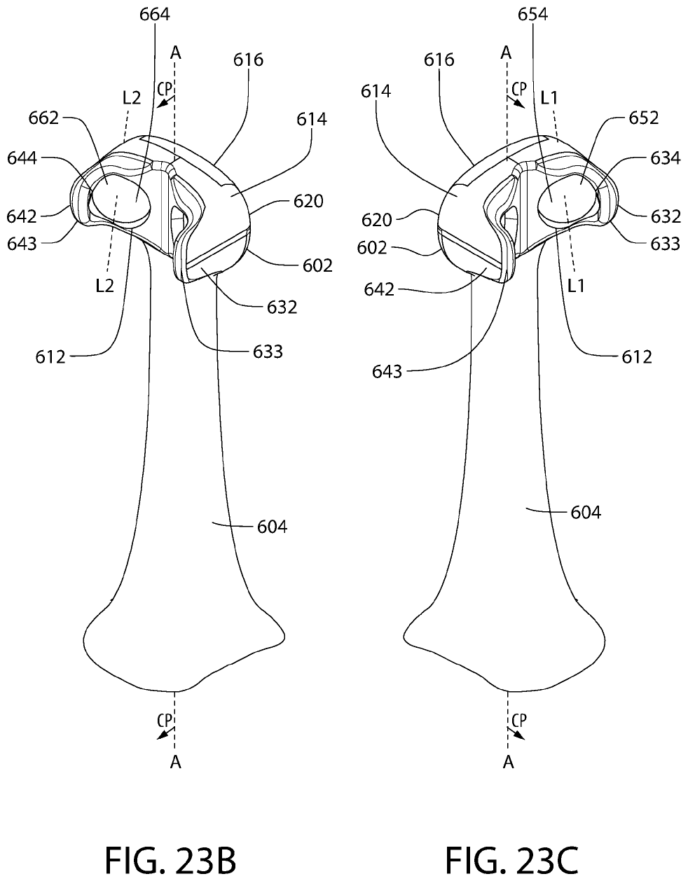

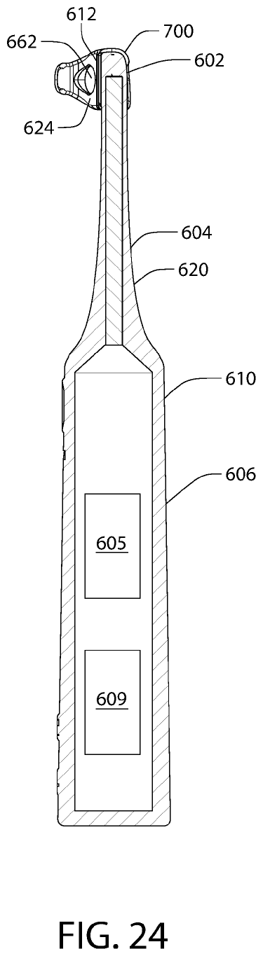

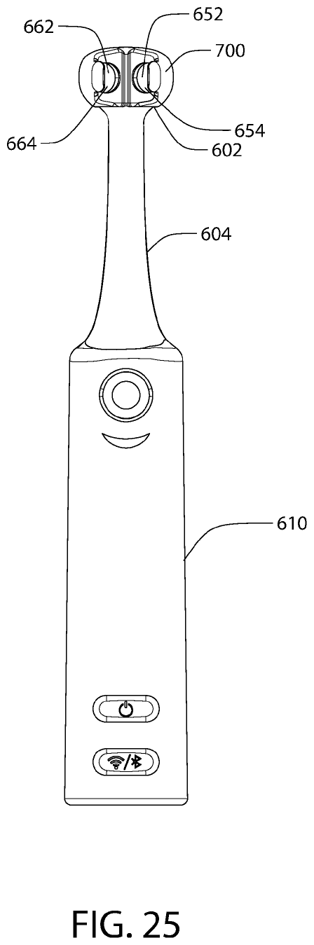

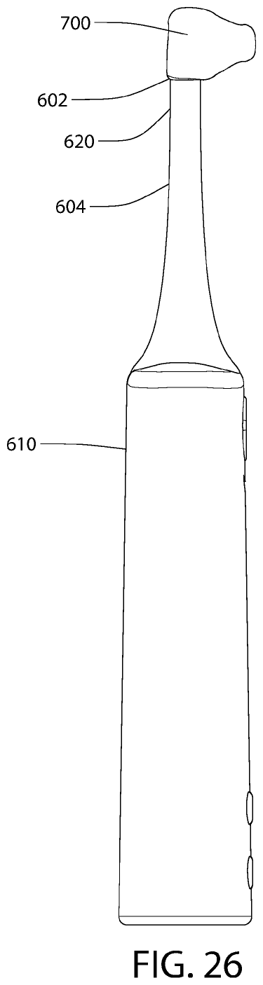

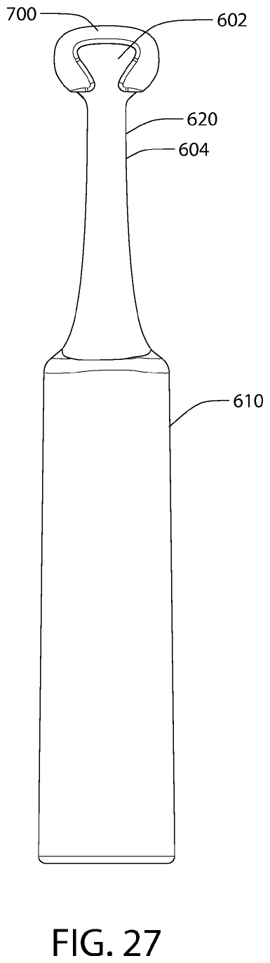

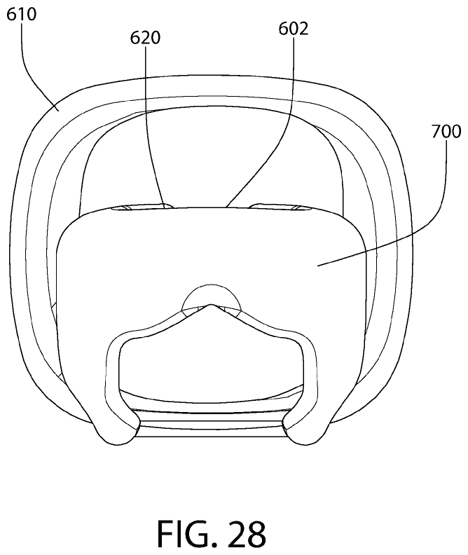

FIGS. 23A-28 illustrate a sixth embodiment illustrating an oral health monitoring device 600. The oral health monitoring device 600 is analogous to the device 100 except as noted. The device 600 generally has a body 610 and a scan head 620. Optionally, the scan head 620 may be detachably coupled to the body 610. More specifically, the body 610 has a handle portion 606 that is configured for gripping and handling by a user. The handle portion 606 may include a plurality of user input devices 607 which may be buttons or switches configured to receive input from a user. The handle portion 606 may also include a plurality of indicators 608 which provide indications to the user. In some implementations, the user input devices 607 and the indicators 608 may be combined such as in a touch screen or buttons which incorporate illumination. A sleeve 700 covers the scan head 620.

The scan head 620 has an elongate body 604 and a head 602. The scan head 620 may be detachably coupled to the body 610 with a friction/interference fit or via mechanical interaction, such as the scan head 620 having a protuberance or recess that matches with a recess or protuberance on the body 610. Various techniques for coupling a scan head 620 to a body 610 of a toothbrush 600 are known and could be used in accordance with the invention described herein (i.e., magnetic, mechanical, interference, screw threads, protuberance/detent, or the like). In other implementations, the scan head 620 and the body 610 may be integrally formed and unitary such that scan head 620 is not detachably coupled to the body 610. The scan head 620 and the body 610 are illustrated generically and the invention is not to be limited by the shape, size, and/or geometry of these components.

The elongate body 604 of the scan head 620 extends along a longitudinal axis A-A. A central plane CP extends through the head 602, the central plane CP defined by the longitudinal axis A-A and extending perpendicular to a front surface 612 of a base portion 614 of the head 602. The base portion 614 also has a rear surface 616 opposite the front surface 612. A first wing structure 632 and a second wing structure 642 extend from the base portion 614. In the present embodiment, the first and second wing structures 632, 642 may be symmetrical about the central plane CP. Otherwise stated, the first and second wing structures 632, 642 are mirrored about the central plane CP. In other embodiments, the first and second wing structures 632, 642 may not be symmetrical about the central plane CP. The first and second wing structures 632, 642 extend to distal ends 633, 643.

The base portion 614 is directly coupled to the elongate body 604. The base portion 614 serves as a central structural feature from which the first and second wing structures 632, 642 extend. A first image sensor 652 is coupled to an inner surface 634 of the first wing structure 632. A second image sensor 662 is coupled to an inner surface 644 of the second wing structure 642. The first and second image sensors 652, 662 are image sensors configured to image optical light. In other embodiments, the first and second image sensors may be sensitive to X-rays, infrared light, UV light, or any other frequency band useful for monitoring oral health.

The first and second image sensors 652, 662 each have imaging surfaces 654, 664. The imaging surfaces 654, 664 may be planar or may convex or concave. Optionally, the imaging surfaces 654, 664 may be lenses or protective windows covering the first and second image sensors 652, 662. A first sensing axis L₁-L₁ is normal to the imaging surface 654 of the first image sensor 652. A second sensing axis L₂-L₂ is normal to the imaging surface 664 of the second image sensor 662. Where the imaging surfaces 654, 664 are concave or convex, the first and second sensing axes L₁-L₁, L₂-L₂ are normal to a plane which is tangent to the imaging surfaces 654, 664 at a minimum or maximum point (as applicable depending on the curvature being concave or convex). The first and second sensing axes L₁-L₁, L₂-L₂ intersect. The first and second sensing axes L₁-L₁, L₂-L₂ intersect at the central plane CP.

The image sensors 652, 662 may be mounted such that they are recessed within or coupled to the inner surfaces 634, 644 of the first and second wing structures 632, 642. Optionally, the image sensors 652, 662 may be recessed on the inner surfaces 634, 644 or the image sensors 652, 662 may be flush mounted or protruding from the inner surfaces 634, 644 of the first and second image sensors 652, 662. A cavity may be formed within the head 602 to contain the image sensors 652, 662 and any associated wiring. In addition, light emitting elements or light guides/light pipes may be incorporated within the cavity as will be discussed below.

The first and second wing structures 632, 642 may be used to maintain a desired spacing between the first and second image sensors 652, 662 and the user's teeth and oral tissues. The desired spacing between the user's teeth and the first and second image sensors 652, 662 may be selected to ensure that the first and second image sensors 652, 662 are positioned at an appropriate focal distance. In some situations, different focal distances may be required due to variations in a user's oral structures or other causes. One of a plurality of sleeves 700 may be utilized, with the sleeves 700 making contact with the user's oral tissues instead of the first and second wing structures 632, 642. As will be discussed in greater detail below, a plurality of sleeves 700 may be utilized on the head 602 to allow optimal spacing between the first and second image sensors 652, 662 and the user's teeth and oral tissues.

In the present embodiment, the inner surfaces 634, 644 of the first and second wing structures 632, 642 may be non-parallel. In other implementations, the inner surfaces 634, 644 may be parallel. The inner surfaces 634, 644 are symmetrical about the central plane CP, but in other implementations the inner surfaces 634, 644 may not be symmetrical about the central plane CP. When viewed from the top of the scan head 620, the base portion 614 and the first and second wing structures 632, 642 form a tooth passageway 624 configured to receive one or more teeth of a user. The tooth passageway 624 has a generally U shape in cross-section.

Optionally, the wing structures 632, 642 may be formed at least in part of soft materials such as elastomers to provide a comfortable interface between the scan head 620 and the user's teeth and gums. Optionally, the soft material may be a thermoplastic elastomer (“TPE”) which may form an entirety of the wing structures 632, 642. In yet other configurations, soft material may be used to form only a portion of the wing structures 632, 642.

The first and second image sensors 652, 662 may incorporate lights to illuminate the user's oral tissues. Optionally, the lights may be ring lights forming a perimeter of the first and second image sensors 652, 662. In other configurations, the lights may be embedded in the wing structures 632, 642 and physically separate from the remainder of the first and second image sensors 652, 662. Optionally, the lights may be LEDs (light emitting diodes) which may be directly mounted on the wing structures 632, 642 or may be embedded, with light pipes or light guides used to direct light from the LEDs to the user's oral tissues. The lights or the light guides may also incorporate a diffuser which may be embedded within the wing structures 632, 642, the diffuser providing an even field of illumination to the user's oral tissues to aid in imaging.

Optionally, the body 610 may further incorporate an energy storage device 609 and an electronic circuit 605, the energy storage device 609 powering the electronic circuit 605. The electronic circuit may incorporate a processor and a memory. The electronic circuit 605 may be electrically connected to the image sensors 652, 662. For instance, the electronic circuit 605 may be activated in response to inputs received by the user input devices 607. The electronic circuit 605 may then record images from the image sensors 652, 662 and store them in the memory for later evaluation. The electronic circuit 605 may also transmit them through one or more means, including wired or wireless communication, either later or immediately upon capturing the images. Any known storage and transmission means may be used. For instance, the images may be transmitted using Wi-Fi, Bluetooth, USB, and other analogous technologies and standards. In alternate implementations, the processor of the electronic circuit 605 may be used to perform on-board analysis of the images to identify areas of interest, including areas having plaque, cavities, or other oral health conditions that may be of interest.

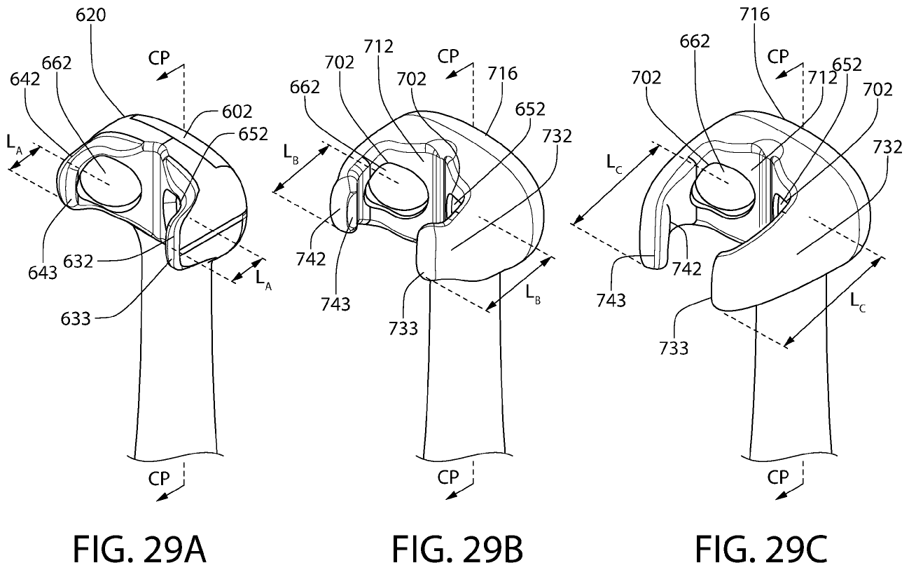

Turning to FIGS. 29A-C, the scan head 620 of the oral health monitoring device 600 is illustrated in a variety of configurations. In FIG. 29A, the scan head 620 is illustrated without a sleeve 700 as discussed above. In this implementation, the scan head 620 may position the image sensors 652, 662 closest to the user's oral tissues as a result of the focal distance being set by a length L_{A} of the first and second wing structures 632, 642. The length L_{A} may be measured from a center of the imaging surfaces 654, 664 adjacent one of the first and second image sensors 652, 662 to the distal ends 633, 643 in a direction parallel to the central plane CP-CP.

Thus, the length L_{A} of the first and second wing structures 632, 642 determines the focal distance between the user's oral tissues and the first and second image sensors 652, 662. Although the user's oral tissues may vary in shape and size, the positioning of the scan head 620 is determined by the length of the first and second wing structures 632, 642. It should be noted that the included angle between the first and second wing structures 632, 642 may be altered in other embodiments, which may also have an impact on the focal length of the first and second image sensors 652, 662.

FIG. 29B illustrates a sleeve 700 on the head 602 of the scan head 620. As discussed above, the sleeve 700 covers the head 602. Optionally, the sleeve 700 may be formed of an elastomeric or rubber material such as a thermoplastic elastomer. The sleeve 700 may be configured to snap over the head 602 without the need for separate fasteners, instead relying on the flexibility of the sleeve 700 to allow installation and removal on the head 602. The sleeve 700 has two apertures 702 which ensure that the image sensors 652, 662 are not obstructed. The apertures 702 are approximately the same size as the imaging surface 654, 664 to ensure that the light sources and the image sensors 652, 662 can pass light through the apertures 702.

The sleeve 700 has a front surface 712, a rear surface 716, a first wing structure 732, and a second wing structure 742. The first and second wing structures 732, 742 extend the first and second wing structures 632, 642 of the head 602, increasing a distance L_{B} as compared with the distance L_{A}. The distance L_{B} is measured from a center of the imaging surfaces 654, 664 adjacent one of the first and second image sensors 652, 662 to distal ends 733, 743 of the first and second wing structures 732, 742 in a direction parallel to the central plane CP-CP.

The first and second wing structures 732, 742 of the sleeve 700 curve around toward the central plane CP-CP. This is done to engage the user's oral tissues more effectively. The total curvature of the wing structure 732, 742 is greater than the wing structures 632, 642 of the head 602, but the spacing between the distal ends 733, 743 of the first and second wing structures 732, 742 may be the same, greater, or less than the spacing between the distal ends 633, 643 of the wing structures 632, 642. It is generally preferable to maintain approximately the same spacing between the distal ends 733, 743, 633, 643 regardless of whether the sleeve 700 is installed on the head 602, so the curvature of the sleeve 700 may be different than the curvature of the wing structures 632, 642 of the head 602.

The distal ends 733, 743, 633, 643 engage the user's oral tissues. The addition of the sleeve 700 alters the spacing between the user's oral tissues and the image sensors 652, 662, allowing alteration of the focal distance by adding or removing the sleeve 700. This allows adjustment for a variety of users without a need for adjustment of the design for varying oral tissues, or compromising focus or other optical characteristics to ensure adequate focus for all users.

Turning to the scan head 620 of FIG. 29C, a second sleeve 700 is illustrated coupled to the head 602. The second sleeve 700 is numbered identically to the first sleeve 700 discussed in FIG. 29B except as noted. Optionally, the second sleeve 700 may be formed of an elastomeric or rubber material such as a thermoplastic elastomer. The second sleeve 700 may be configured to snap over the head 602 without the need for separate fasteners, instead relying on the flexibility of the sleeve 700 to allow installation and removal on the head 602. The second sleeve 700 has two apertures 702 which ensure that the image sensors 652, 662 are not obstructed. The apertures 702 are approximately the same size as the imaging surface 654, 664 to ensure that the light sources and the image sensors 652, 662 can pass light through the apertures 702.

The sleeve 700 has a front surface 712, a rear surface 716, a first wing structure 732, and a second wing structure 742. The first and second wing structures 732, 742 extend the first and second wing structures 632, 642 of the head 602, increasing a distance Lc as compared with the distances L_{A}, L_{B}. The distance L_{C} is measured from a center of the imaging surfaces 654, 664 adjacent one of the first and second image sensors 652, 662 to distal ends 733, 743 of the first and second wing structures 732, 742 in a direction parallel to the central plane CP-CP.

The first and second wing structures 732, 742 of the sleeve 700 curve around toward the central plane CP-CP. This is done to engage the user's oral tissues more effectively. The total curvature of the wing structure 732, 742 is greater than the wing structures 632, 642 of the head 602, but the spacing between the distal ends 733, 743 of the first and second wing structures 732, 742 may be the same, greater, or less than the spacing between the distal ends 633, 643 of the wing structures 632, 642. It is generally preferable to maintain approximately the same spacing between the distal ends 733, 743, 633, 643 regardless of whether the sleeve 700 is installed on the head 602, so the curvature of the sleeve 700 may be different than the curvature of the wing structures 632, 642 of the head 602.

The distal ends 733, 743, 633, 643 engage the user's oral tissues. The addition of the sleeve 700 alters the spacing between the user's oral tissues and the image sensors 652, 662, allowing alteration of the focal distance by adding or removing the sleeve 700. This allows adjustment for a variety of users without a need for adjustment of the design for varying oral tissues, or compromising focus or other optical characteristics to ensure adequate focus for all users. As can be seen, a variety of sleeves 700 may be provided, allowing selection of the optimal focal length for a given user.

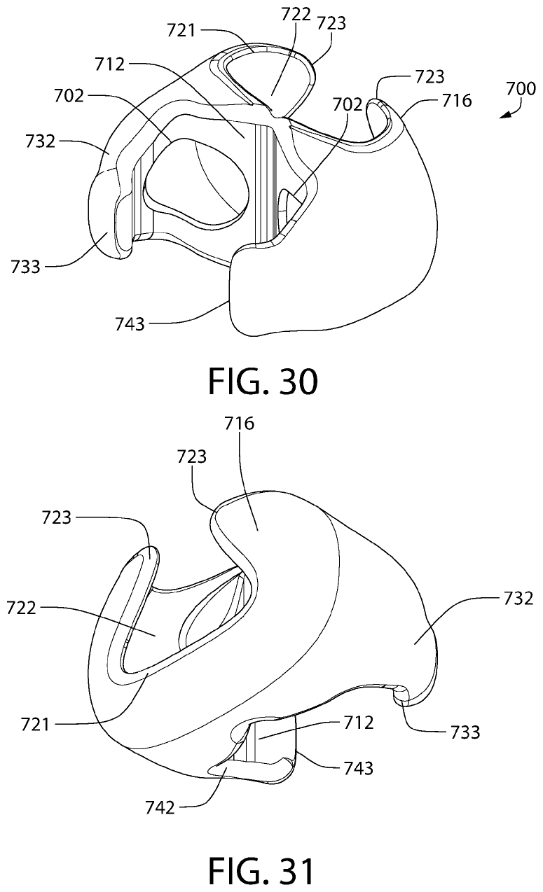

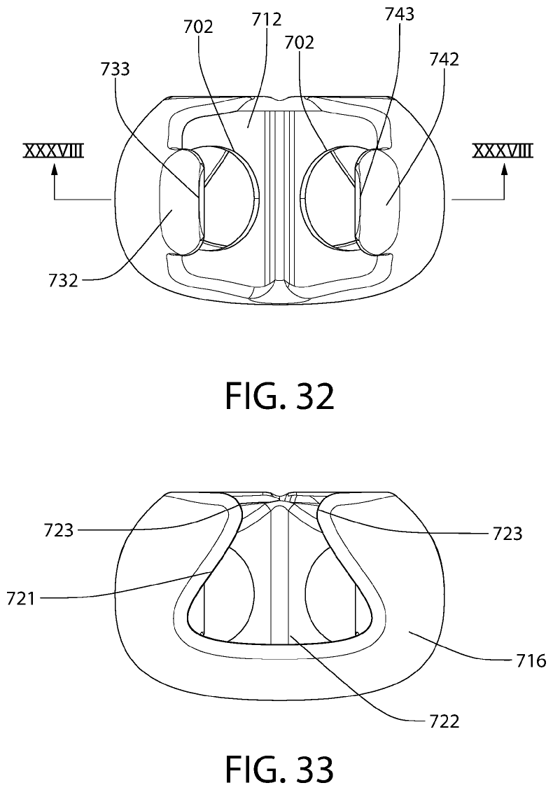

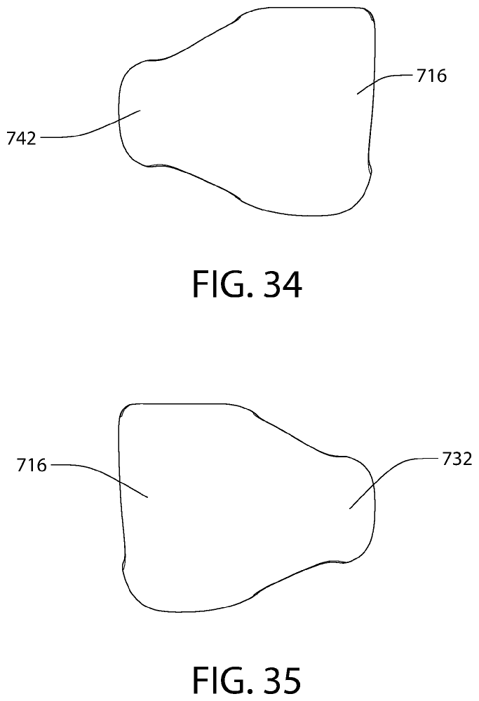

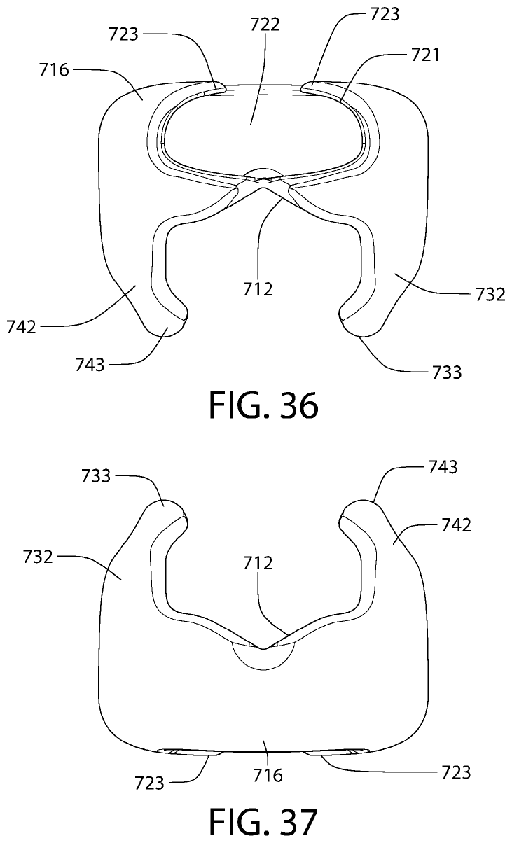

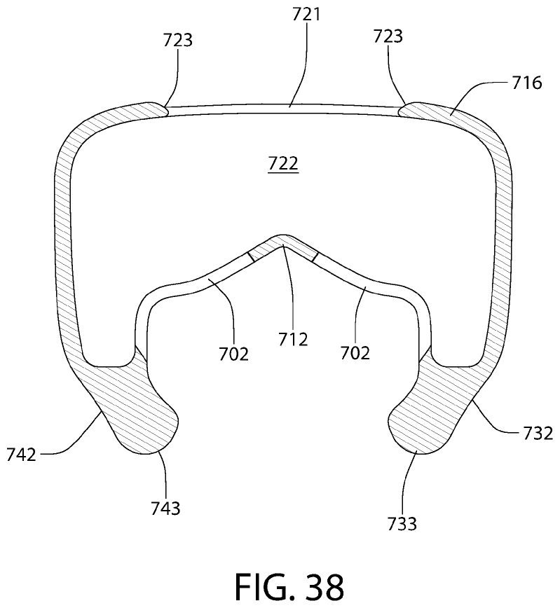

FIGS. 30-38 illustrate a sleeve 700 in greater detail. The sleeve 700 has a front surface 712 and a rear surface 716, with two apertures 702 in the front surface 712 to expose the first and second image sensors 652, 662. A head opening 721 extends to a cavity 722 that receives the head 602. The sleeve 700 further incorporates a plurality of retention elements 723 that are configured to surround the head 602 and maintain the head 602 within the cavity 722. The retention elements 723 may form a portion of the head opening 721 or may be separately formed. Similarly, the head opening 721 may incorporate one or more sealing features which provide a liquid-tight seal between the head 602 and the sleeve 700.

The sleeve 700 further incorporates first and second wing structures 732, 742 that extend to distal ends 733, 743. The first and second wing structures 732, 742 are symmetrical, although in other implementations the first and second wing structures 732, 742 may not be symmetrical. The wing structures 732, 742 may be at least partially hollow, with the cavity 722 extending into a portion of the first and second wing structures 732, 742. As noted above, the sleeve 700 may be formed of an elastomeric material to promote flexibility and ensure user comfort.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

1. An oral health monitoring device, the device comprising:
an elongate body extending along a longitudinal axis; and
a head coupled to the elongate body, the head comprising:
a base portion, the base portion coupled to the elongate body;
a first wing structure extending from the base portion;
a second wing structure extending from the base portion;
a first image sensor coupled to the first wing structure, the first image sensor having a first sensing axis normal to an imaging surface of the first image sensor; and
a second image sensor coupled to the second wing structure, the second image sensor having a second sensing axis normal to an imaging surface of the second image sensor;
wherein the first and second sensing axes of the first and second image sensors intersect.

2. The device of claim 1 wherein the first wing structure and the second wing structure each has an inner surface, the inner surfaces of the first and second wing structures being non-parallel.

3. The device of claim 1 wherein the head further comprises a tooth guide protruding from the base portion, the tooth guide configured to engage a tooth of a user and isolate the base portion from the tooth of the user.

4. The device of claim 1 further comprising a central plane, the longitudinal axis extending along the central plane and the central plane extending perpendicular to a front surface of the base portion.

5. The device of claim 4 wherein the first and second wing structures are symmetrical about the central plane.

6. The device of claim 4 wherein the head further comprises a tooth guide protruding from the front surface of the base portion, the tooth guide extending along the central plane.

7. The device of claim 6 wherein the tooth guide is symmetrical about the central plane.

8. The device of claim 1 wherein the first wing structure extends perpendicular to the longitudinal axis and the second wing structure extends at an oblique angle to the longitudinal axis.

9. The device of claim 1 wherein the first and second wing structures and the base portion collectively form a tooth passageway, the tooth passageway having a U shape.

10. The device of claim 1 wherein the base portion comprises a cavity, a tooth guide forming a wall of the cavity.

11. The device of claim 10 wherein one of the first or second image sensors is mounted within the cavity.

12. The device of claim 1 further comprising a sleeve, the sleeve configured to cover the head.

13. The device of claim 12 wherein the sleeve comprises first and second wing extensions, the first and second wing extensions extending from the first and second wing structures.

14. An oral health monitoring device, the device comprising:
an elongate body extending along a longitudinal axis; and
a head coupled to the elongate body, the head comprising:
a base portion, the base portion coupled to the elongate body;
a first wing structure extending from the base portion, the first wing structure having an inner surface;
a second wing structure extending from the base portion, the second wing structure having an inner surface;
a first image sensor coupled to the first wing structure; and
a second image sensor coupled to the second wing structure;
wherein the inner surfaces of the first and second wing structures are non-parallel.

15. The device of claim 14 wherein the first image sensor has a first sensing axis normal to an imaging surface of the first imaging sensor and the second image sensor has a second sensing axis normal to an imaging surface of the second imaging sensor, the first and second sensing axes of the first and second image sensors intersecting.

16. The device of claim 14 wherein the head further comprises a tooth guide protruding from the base portion, the tooth guide configured to engage a tooth of a user and isolate the base portion from the tooth of the user.

17.-20. (canceled)

21. The device of claim 14 wherein the first wing structure extends perpendicular to the longitudinal axis and the second wing structure extends at an oblique angle to the longitudinal axis.

22. (canceled)

23. The device of claim 14 wherein the base portion comprises a cavity, a tooth guide forming a wall of the cavity.

24. The device of claim 23 wherein one of the first or second image sensors is mounted within the cavity.

25. (canceled)

26. (canceled)

27. An oral health monitoring device, the device comprising:
an elongate body extending along a longitudinal axis; and
a head coupled to the elongate body, the head comprising:
a base portion, the base portion coupled to the elongate body;
a first wing structure extending from the base portion, the first wing structure having an inner surface;
a second wing structure extending from the base portion, the second wing structure having an inner surface;
a first image sensor coupled to the first wing structure;
a second image sensor coupled to the second wing structure; and
a tooth guide extending from the base portion, the tooth guide configured to engage a tooth of a user and isolate the base portion from the tooth of the user.

28.-39. (canceled)
